(19) United States
(12) Patent Application Publication
(10) Pub. No.: US 2025/0277009 A1
(43) Pub. Date: Sep. 4, 2025

(54) HHIP-FC FUSION PROTEINS AND USES THEREOF

(71) Applicant: The Regents of the University of California, Oakland, CA (US)

(72) Inventor: Tien Peng, San Francisco, CA (US)

(73) Assignee: The Regents of The University of California, Oakland, CA (US)

(21) Appl. No.: 18/858,970

(22) PCT Filed: Apr. 24, 2023
(86) PCT No.: PCT/US2023/019568

§ 371 Date: Oct. 22, 2024

Related U.S. Application Data
(60) Provisional application No. 63/335,165, filed on Apr. 26, 2022.

(51) Int. Cl. C07K 14/47; A61K 38/00; A61P 11/06

(52) U.S. Cl. CPC C07K 14/4703; A61P 11/06; A61K 38/00; C07K 2319/30

(57) ABSTRACT

Compositions comprising Hedgehog-binding portion of a human Hedgehog Interacting Protein (HHIP) as well as their use in inhibiting inflammation in tissues are provided.

### CROSS-REFERENCE TO RELATED PATENT APPLICATIONS

The present application claims benefit of priority to U.S. Provisional Patent Application No. 63/335,165, filed Apr. 26, 2022, which is incorporated by reference for all purposes.

### STATEMENT AS TO RIGHTS TO INVENTIONS MADE UNDER FEDERALLY SPONSORED RESEARCH AND DEVELOPMENT

This invention was made with government support under grant no. R01 HL142552 awarded by The National Institutes of Health. The government has certain rights in the invention.

### BACKGROUND OF THE INVENTION

A fundamental adaptation of barrier epithelia is the ability to repel environmental pathogens by hosting resident immune cells that can respond to diverse infectious and environmental insults. In particular, the establishment of lymphocyte residency integrates the innate and adaptive immune response at the barrier, where innate lymphoid cells (ILCs), unconventional T cells, and CD4+ and CD8+ tissue-resident memory T cells (TRMs) are uniquely positioned to act as first-responders to common and repetitive stimuli. While the antimicrobial benefits of these tissue-resident lymphocytes (TRLs) are well-established, the cost of their tissue residency to the host organ is less clear. This is particularly relevant in the lung, where the barrier epithelia must not only limit microbial invasion but also allow for gas exchange. Indeed, repetitive infections with air-borne microbes have been linked to the pathogenesis of chronic lung diseases characterized by excessive inflammation such as chronic obstructive pulmonary disease (COPD).

COPD is a highly prevalent and incurable lung disease affecting close to 10% of adults globally. COPD represents a spectrum of lung dysfunctions ranging from airway inflammation (chronic bronchitis) to loss of respiratory epithelium (emphysema). The typical clinical course of COPD is characterized by periods of symptomatic stability punctuated by acute exacerbations that lead to step-wise and irreversible decline in lung function. Bronchoalveolar lavage studies implicate viral respiratory tract infections as the major trigger for acute exacerbations. Thus, resolving the mechanistic link between acute infectious exacerbations and loss of lung function could reveal targeted approaches to treat COPD, moving beyond current treatments with corticosteroids that have broad systemic side-effects and do little to halt disease progression.

Hedgehog interacting protein (HHIP) is highly expressed in the lung, and the HHIP locus has been repeatedly identified in large genome wide association studies as a disease-susceptibility locus for COPD (Pillai et al., Am J Respir Crit Care Med. 2010 Dec. 15;182 (12): 1498-505). HHIP is a negative regulator of hedgehog signaling, a pathway that regulates lung stromal—epithelial crosstalk. Further, recent studies suggest the presence of stromal niches within the lung that locally regulate accumulation and function of diverse TRLs. However, there are no known classes of drugs or biologic agents that specifically target TRLs and prevent their accumulation in the lung, which could have beneficial effect on chronic lung diseases where lung inflammation is implicated.

### BRIEF SUMMARY OF THE INVENTION

In some embodiments, the disclosure provides a polypeptide comprising at least one soluble Hedgehog-binding portion of a human Hedgehog Interacting Protein (HHIP) linked to a molecule that enhances blood half-life of the polypeptide.

In some embodiments, the portion comprises SEQ ID NO: 1 or an amino acid sequence at least 90 or 95 or 95% identical thereto. In some embodiments, the portion comprises SEQ ID NO: 2 or an amino acid sequence at least 90 or 95 or 99% identical thereto.

In some embodiments, the polypeptide comprises two soluble Hedgehog-binding portions of a HHIP. In some embodiments, each of the two soluble Hedgehog-binding portions comprise SEQ ID NO: 1 or an amino acid sequence at least 90 or 95 or 99% identical thereto. In some embodiments, the two soluble Hedgehog-binding portions are linked to each other via a linker sequence. In some embodiments, the linker sequence comprises SEQ ID NO:8.

In some embodiments, the at least one soluble Hedgehog-binding portion is linked via an amino acid linker to the molecule. In some embodiments, the amino acid linker comprises glycine and serine. In some embodiments, the amino acid linker comprises SEQ ID NO:3.

In some embodiments, the polypeptide comprises SEQ ID NO:7 or an amino acid sequence at least 90 or 95 or 99% identical thereto.

In some embodiments, the molecule is an Fc portion of an antibody. In some embodiments, the Fc portion comprises SEQ ID NO: 4 or an amino acid sequence at least 90 or 95 or 99% identical thereto.

In some embodiments, the polypeptide comprises SEQ ID NO: 5 or SEQ ID NO:6 or an amino acid sequence at least 90 or 95 or 99% identical thereto.

In some embodiments, the molecule is polyethylene glycol.

Also provided is a polynucleotide encoding the polypeptide as described above or elsewhere herein.

Also provided is an expression cassette or expression vector comprising a promoter operably linked to the polynucleotide as described above or elsewhere herein.

Also provided is a host cell comprising the expression cassette or expression vector as described above or elsewhere herein. A host cell can be for example a prokaryotic cell (which can include but is not limited to, E. coli) or a eukaryotic cell (which can include but is not limited to, a fungal, yeast, insect, plant or mammalian (e.g., human) cell).

A dimer comprising two polypeptides, each polypeptide comprising at least one soluble Hedgehog-binding portion of a human Hedgehog Interacting Protein (HHIP) linked to an Fc portion of an antibody, wherein the Fc portions of the two polypeptides are linked by one or more disulfide bond. The two polypeptides can be identical copies of they can have different amino acid sequences. In some embodiments, the portions comprise SEQ ID NO: 1 or an amino acid sequence at least 90 or 95 or 95% identical thereto. In some embodiments, the portion comprises SEQ ID NO:2 or an amino acid sequence at least 90 or 95 or 99% identical thereto. In some embodiments, one or both o the polypeptides comprise two soluble Hedgehog-binding portions of a HHIP. In some embodiments, each of the two soluble Hedgehog-binding portions comprise SEQ ID NO:1 or an amino acid sequence at least 90 or 95 or 99% identical thereto. In some embodiments, the two soluble Hedgehog-binding portions are linked to each other via a linker sequence. In some embodiments, the linker sequence comprises SEQ ID NO:8. In some embodiments, the at least one soluble Hedgehog-binding portions are linked via an amino acid linker to the molecule. In some embodiments, the amino acid linker comprises glycine and serine. In some embodiments, the amino acid linker comprises SEQ ID NO:3. In some embodiments, one or both polypeptides comprise SEQ ID NO:7 or an amino acid sequence at least 90 or 95%. In some embodiments, the Fc portion comprises SEQ ID NO: 4 or an amino acid sequence at least 90 or 95 or 99% identical thereto. In some embodiments, one or both of the polypeptides comprise SEQ ID NO: 5 or SEQ ID NO:6 or an amino acid sequence at least 90 or 95 or 99% identical thereto.

In some embodiments, the disclosure provides a method of inhibiting Hedgehog signaling in a human in need thereof. In some embodiments, the method comprises administering the polypeptide as described above or elsewhere herein, or the dimer as described above or elsewhere herein, or a nucleic acid comprising the expression cassette as described above or elsewhere herein to the human in an amount sufficient to inhibit Hedgehog signaling.

In some embodiments, the administering comprises administering the polypeptide or nucleic acid orally, intravenously, or via inhalation nasally or orally.

In some embodiments, the human has inflammation in the lungs and administration of the polypeptide or nucleic acid reduces the inflammation.

In some embodiments, the human has chronic obstructive pulmonary disease (COPD), pulmonary fibrosis, asthma, lung cancer, graft-versus host disease of a lung transplant, follicular bronchiolitis or interstitial lung disease.

### Definitions

As used in this specification and the appended claims, the singular forms “a,” “an,” and “the” include plural referents unless the context clearly dictates otherwise.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) as well as pyrrolysine, pyrroline-carboxy-lysine, and selenocysteine.

The term “conservative amino acid substitutions” refers to the substitution (conceptually or otherwise) of an amino acid from one such group with a different amino acid from the same group. One example of substitutions is based on analyzing the normalized frequencies of amino acid changes between corresponding proteins of homologous organisms (see, e.g., Schulz, G. E. and R. H. Schirmer, Principles of Protein Structure, Springer-Verlag). According to such analyses, groups of amino acids may be defined where amino acids within a group exchange preferentially with each other and, therefore, resemble each other most in their impact on the overall protein structure (see, e.g., Schulz, G. E. and R. H. Schirmer, Principles of Protein Structure, Springer-Verlag). One example of a set of amino acid groups defined in this manner include: (i) a charged group, consisting of Glu and Asp, Lys, Arg and His; (ii) a positively-charged group, consisting of Lys, Arg and His; (iii) a negatively-charged group, consisting of Glu and Asp; (iv) an aromatic group, consisting of Phe, Tyr and Trp; (v) a nitrogen ring group, consisting of His and Trp; (vi) a large aliphatic nonpolar group, consisting of Val, Leu and Ile; (vii) a slightly-polar group, consisting of Met and Cys; (viii) a small-residue group, consisting of Ser, Thr, Asp, Asn, Gly, Ala, Glu, Gln and Pro; (ix) an aliphatic group consisting of Val, Leu, Ile, Met and Cys; and (x) a small hydroxyl group consisting of Ser and Thr. Other examples of conservative substitutions based on shared physical properties are the substitutions within the following groups: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

The terms “peptidomimetic” and “mimetic” refer to a synthetic chemical compound that has substantially the same functional characteristics of a naturally or non-naturally occurring polypeptide, but different (though typically similar) structural characteristics. Peptide analogs are commonly used in the field as non-peptide active compounds (e.g., drugs) with properties analogous to those of a template peptide. Such non-peptide compounds are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987)). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as found in a polypeptide of interest, but have one or more peptide linkages optionally replaced by a linkage selected from the group consisting of, e.g., —CH2NH—, —CH2S—, —CH2-CH2-, —CH═CH— (cis and trans), —COCH2-, —CH(OH)CH2-, and —CH2SO—. A mimetic can be either entirely composed of synthetic, non-natural analogues of amino acids, or, is a chimeric molecule of partly natural peptide amino acids and partly non-natural analogs of amino acids. A mimetic can also incorporate any amount of natural amino acid conservative substitutions as long as such substitutions also do not substantially alter the mimetic's structure and/or activity.

“Percentage of sequence identity” is determined by comparing two optimally aligned sequences over a comparison window, wherein the portion of the amino acid sequence or polynucleotide sequence in the comparison window may comprise additions or deletions (i.e., gaps) as compared to the reference sequence (e.g., SEQ ID NO:1: 2, 4, 5, 6, or 7), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 95% identity, optionally 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. For an amino acid sequence, optionally, identity exists over a region that is at least about 50 amino acids in length, or more preferably over a region that is 100 to 150 or 200 or more amino acids in length, or where not indicated over the entire length of the reference sequence.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 50 to 600, usually about 75 to about 200, more usually about 100 to about 150 in which a sequence may be compared to a reference sequence of the same number of contiguous positions after the two sequences are optimally aligned. Methods of alignment of sequences for comparison are well known in the art.

An algorithm for determining percent sequence identity and sequence similarity is the BLAST 2.0 algorithms, e.g., as described in, and Altschul et al. (1990) J. Mol. Biol. 215:403-410 (see also Altschul et al. (1977) Nuc. Acids Res. 25:3389-3402). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, and N=−4.

The term “isolated,” when applied to a nucleic acid or protein, denotes that the nucleic acid or protein is purified to be essentially free of other cellular components with which it is associated in the natural state. It is often in a homogeneous or nearly homogeneous state. It can be in either a dry or aqueous solution. Purity and homogeneity may be determined using analytical chemistry techniques known and used typically in the art, e.g., polyacrylamide gel electrophoresis, high performance liquid chromatography, etc. A protein that is the predominant species present in a preparation is substantially purified. The term “purified” in some embodiments denotes that a protein gives rise to essentially one band in an electrophoretic gel. Typically, it means that a protein is at least 85% pure, e.g., at least 95% pure, or at least 99% pure.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

As used herein, a “soluble portion” of HHIP refers to a fragment of HHIP that comprises at least a portion of the extracellular domain of HHIP and does not include the HHIP transmembrane portion and thus is soluble in aqueous solutions. In some embodiments, the portion of the extracellular domain can include, for example, at least 50, 100, 150, 200, 300, or 400 or more contiguous amino acids of the human HHIP or of SEQ ID NO:1.

### BRIEF DESCRIPTION OF THE DRAWINGS

FIG. 1 depicts structural features of the human HHIP protein with positions of amino acids noted.

FIG. 2 depicts various embodiments of portions of the human HHIP protein fused to an FC domain. At the left, the entire soluble portion of HHIP is fused to an Fc domain. In the middle, only the 6-bladed β-propeller domain is fused to an FC domain. At the right, two copies of the 6-bladed β-propeller domain of HHIP are fused to an Fc domain. In each depicted embodiments, the fusion proteins form a dimer by disulfide bonding between the two Fc domains.

FIG. 3 depicts the effect of various Fc fusions on Hedgehog activation.

FIG. 4 depicts the effect of HHIP-Fc (Seq ID NO:5, Version 1) is specific in suppressing tissue-resident lymphocytes (TRLs) but not circulating (systemic) lymphocytes (CLs) in the lung after respiratory viral infection.

FIG. 5 depicts truncated version of HHIP-Fc (Seq ID NO:6, Version 2) effect on tissue-resident lymphocytes (TRLs) in the lung.

FIG. 6 depicts the effect of HHIP-Fc (SEQ ID NO:5, Version 1) in reducing airspace enlargement seen in an emphysema animal model compared to IgG isotype control.

FIG. 7 depicts the effect of HHIP-Fc (SEQ ID NO:5, Version 1) vs IgG isotype control on the accumulation of IL17+ tissue-resident lymphocytes (TRLs) in the lung after inhalation of the allergen, house dust mite (HDM).

FIG. 8 depicts the effect of HHIP-Fc (SEQ ID NO:5, Version 1) vs IgG isotype control on airway hyperresponsiveness (AHR) after inhalation of the allergen, house dust mite (HDM).

FIG. 9 depicts the effect of HHIP-Fc (SEQ ID NO:5, Version 1) vs IgG isotype control on gas exchange as measured by SaO2 in mouse lungs with existing fibrosis induced by bleomycin.

FIG. 10 depicts the effect of HHIP-Fc (SEQ ID NO:5, Version 1) vs IgG isotype control on fibrotic burden/collagen content as measured by hydroxyproline content in mouse lungs with existing fibrosis induced by bleomycin.

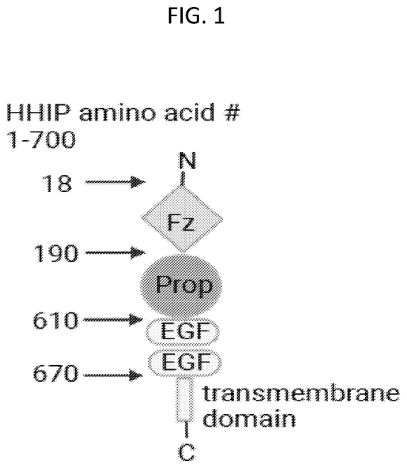

### DETAILED DESCRIPTION OF THE INVENTION

The inventor has discovered that administration of a soluble portion of the Hedgehog-binding portion of a human Hedgehog Interacting Protein (HHIP) can inhibit inflammation by targeting tissue-resident lymphocyte accumulation, for example in the lungs. Moreover, polypeptide constructs have been developed portions of HHIP that bind with Hedgehog and that can be fused to molecules that enhance half-life of the polypeptide following administration to an animal. Exemplary fusion molecules can include, for example, an Fc portion of an antibody.

The disclosure provides for soluble portions of human HHIP that bind to Hedgehog and that can be fused to molecules to extend the half-life of the polypeptide upon administration to an animal, e.g., a human. The inventor has identified several amino-terminal portions of HHIP that are effective in binding Hedgehog and can be fused to half-life extending molecules. For example, SEQ ID NO: 1 represents the sequence of human HHIP from Gly 190-Arg610 (where numbering starts at the first amino acid of the human HHIP). In addition, the inventor determined that the larger portion depicted in SEQ ID NO:2 can also be used. SEQ ID NO:2 represents the sequence of human HHIP from Phe18-Asp670. However, it will be appreciated that other portions of human HHIP can be used so long as the portions are capable of binding Hedgehog. For example, in some embodiments, the portion from HHIP comprises (i.e., can include additional HHIP or non-HHIP amino acids) or consists of (i.e., the portion can be fused to other non-HHIP sequences but does not include other HHIP sequences) SEQ ID NO:1 or SEQ ID NO: 2, but also includes other contiguous amino acids from the extracellular region of the human HHIP. In other embodiments, the portion can comprise or consist of a fragment of SEQ ID NO: 1. One can measure competitive binding to human Hedgehog protein (i.e., as used herein, to measure Hedgehog-binding) based on the ability of HHIP to inhibit hedgehog ligand activation in vitro. Previous crystal structure study has revealed that the 6-bladed β-propeller domain of the HHIP protein is involved in hedgehog ligand binding, and mutations in Asp383 of the propeller domain abolishes HHIP's affinity for the hedgehog ligand (Bishop et al., Nat Struct Mol Biol, 16 (7): 698-703 (2009)).

As noted above, in some embodiments, the HHIP soluble polypeptide is present as part of a fusion protein, e.g., a translational fusion protein comprising an HHIP soluble polypeptide described herein and an Fc polypeptide. As used herein, the term “Fc polypeptide” refers to the C-terminal region of an immunoglobulin heavy chain polypeptide, i.e., lacking an antibody variable region. An Fc polypeptide typically contains constant region sequences (e.g., the CH2 domain and/or the CH3 domain) and may also contain the hinge region (or a portion thereof). In some embodiments, the Fc polypeptide is a naturally-occurring Fc polypeptide. Alternatively, the Fc can include one or more introduced amino acid mutation to improve its properties. As a non-limiting example, positions 250, 314, and 428 can be substituted with another amino acid which is different from that present in the unmodified Fc-fusion protein, thereby altering the binding affinity for FcRn and/or the serum half-life in comparison to the unmodified Fc-fusion protein. See, e.g., U.S. Pat. No. 8,624,007. Methods for fusing or conjugating polypeptides to the constant regions of antibodies (i.e. making Fc fusion proteins) are described in, e.g., U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; WO2011062859; Ashkenazi et al., Proc. Natl. Acad. Sci. USA 88:10535-10539 (1991); Traunecker et al., Nature 331:84-86 (1988); Zheng et al., J. Immunol. 154:5590-5600 (1995); and Vil et al., Proc. Natl. Acad. Sci. USA 89:11337-11341 (1992). An Fc polypeptide typically does not contain a variable region. In some embodiments, the Fc polypeptide is an IgG1, IgG2, IgG3, or IgG4 Fc polypeptide. An exemplary FC polypeptide can include, for example, SEQ ID NO:4.

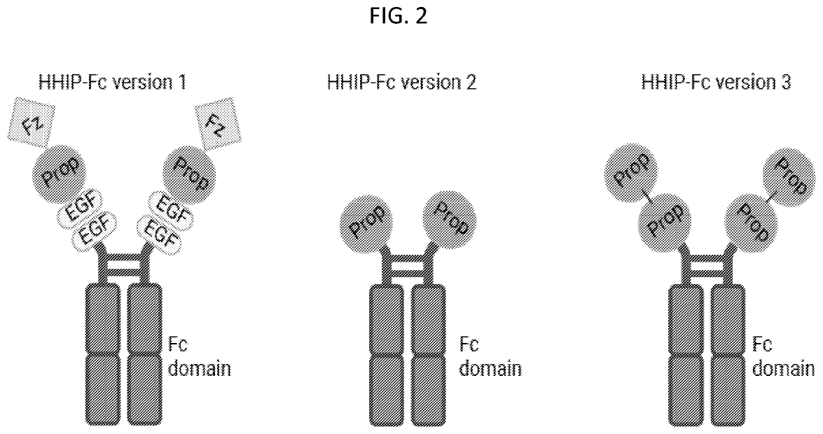

The HHIP soluble polypeptide can be linked directly to the Fc polypeptide or alternatively via an amino acid linker. In some embodiments, the amino acid linker is between 1-30 amino acids long. Any type of linker can be used, but generally the linker will be “flexible” for example formed mainly or exclusively from glycine and serine residues. Exemplary amino acid linker sequences can comprise, for example GGGGS or SEQ ID NO:3 or SEQ ID NO:8. Examples of polypeptides a HHIP soluble portion linked to an Fc portion is depicted in FIG. 2 (left two embodiments) (for example, SEQ ID NO:5 or 6).

In some embodiments, the polypeptides described herein comprise two or three or more HHIP soluble portions, which may be identical or non-identical to each other, which are linked to a molecule to increase half-life in a subject as described herein. In some embodiments, increasing the number of HHIP soluble portions will allow the resulting protein to bind Hedgehog more efficiently. These soluble portions can be linked directly together, or as with linkage to the Fc polypeptide, via an amino acid linker. Exemplary non-limiting linkers can comprise, for example, GGGGS or SEQ ID NO:3 or 8. An example of a polypeptide comprising two HHIP soluble portions linked to an Fc portion is depicted in FIG. 2 (for example, SEQ ID NO: 7).

In some embodiments, a modification such as PEGylation or myristoylation, or fusion to an Fc polypeptide, increases the half-life (e.g., in the body of a subject such as a mammal) of the polypeptide, as compared to a corresponding HHIP soluble polypeptide that does not have the modification or that is not fused to the Fc polypeptide. Increased half-life can be due to, for example, increased stability (i.e., the polypeptide is more resistant to degradation and/or metabolism) and/or decreased clearance (e.g., renal clearance). In some embodiments, half-life of the modified (e.g., PEGylated and/or myristoylated) polypeptide is increased by at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 60-fold, 70-fold, 80-fold, 90-fold, 100-fold, or more.

Introduction of PEG or long chain polymers of PEG increases the effective molecular weight of the present polypeptides, for example, to prevent rapid filtration into the urine. In some embodiments, a Lysine residue in the polypeptide sequence is conjugated to PEG directly or through a linker. Such linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are well known in the art (described, for example, in Veronese, F. M., et al., Drug Disc. Today 10:1451-8 (2005); Greenwald, R. B., et al., Adv. Drug Deliv. Rev. 55:217-50 (2003); Roberts, M. J., et al., Adv. Drug Deliv. Rev., 54:459-76 (2002)).

The polypeptides described herein are useful for example in reducing inflammation or other undesired immune responses in a human subject. As noted herein, in some embodiments, the human subject has or is at risk for having, and inflammatory response in the lungs. The polypeptides described herein, formulated as a pharmaceutical composition can be administered directly to the lunch (e.g., via inhalation) or indirectly, e.g., intravenously or other administration methods.

Also provided are pharmaceutical compositions comprising the polypeptides described herein comprising the soluble portions of HHIP linked to a molecule that extends the polypeptide's half-life in a human that can be formulated with a pharmaceutically acceptable carrier. The compositions can additionally contain other therapeutic agents that are suitable for treating or preventing a given disorder. Pharmaceutically carriers can enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition as described herein can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, intranasal, inhalational (e.g., intranasally), spinal or epidermal administration (e.g., by injection or infusion). Depending on the route of administration, the active compound, i.e., polypeptide described herein, may be coated in a material to protect the compound from the action of acids and other natural conditions that may inactivate the compound.

The polypeptides described herein, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

In some embodiments, the composition is sterile and fluid. Proper fluidity can be maintained, for example, by use of coating such as lecithin, by maintenance of required particle size in the case of dispersion and by use of surfactants. In many cases, it can be preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions of the present invention. Applicable methods for formulating the antibodies and determining appropriate dosing and scheduling can be found, for example, in Remington: The Science and Practice of Pharmacy, 21^{st} Ed., University of the Sciences in Philadelphia, Eds., Lippincott Williams & Wilkins (2005); and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, and Sustained and Controlled Release Drug Delivery Systems, J. R. Robinson, ed., Marcel Dekker, Inc., New York, 1978, each of which are hereby incorporated herein by reference. Pharmaceutical compositions are preferably manufactured under GMP conditions. Typically, a therapeutically effective dose or efficacious dose of the polypeptides described herein is employed in the pharmaceutical compositions. The polypeptides can be formulated into pharmaceutically acceptable dosage forms by conventional methods known to those of skill in the art. Dosage regimens are adjusted to provide the desired response (e.g., a therapeutic response). In determining a therapeutically or prophylactically effective dose, a low dose can be administered and then incrementally increased until a desired response is achieved with minimal or no undesired side effects. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

In certain embodiments, nucleic acids encoding an HHIP soluble polypeptide as described herein (e.g., linked to a molecule that increases the polypeptide half-life) are used for expression of the polypeptide in vitro (e.g., for production of the polypeptide), ex vivo in vivo. These nucleic acids can be inserted into any of a number of well-known vectors, including viral expression vectors or plasmid-based vectors for the transfection of target cells and organisms. The nucleic acids may be transfected into cells, e.g., ex vivo or in vivo or administered in vivo. For production of the polypeptide, one can use prokaryotic or eukaryotic cells for polypeptide production.

In some embodiments, a nucleic acid constructs encoding a HHIP soluble polypeptide is administered as a purified nucleic acid molecule, for example, as a DNA plasmid-based vector (“naked” DNA). Alternatively, in some embodiments, a nucleic acid construct encoding a HHIP soluble polypeptide can be contained within a viral vector and administered as a viral particle. Viral delivery systems include adenovirus vectors (e.g., Ad2, Ad5, Ad7), adeno-associated viral vectors, herpes simplex viral vectors, retroviral vectors, pox viral vectors (such as vaccinia and avian poxvirus vectors, such as the fowlpox and canarypox vectors), lentiviral vectors, alphavirus vectors, poliovirus vectors, and other positive and negative stranded RNA viruses, viroids, and virusoids, or portions thereof.

Nucleic acids for administration to a subject can be formulated for pharmaceutical administration. While any suitable carrier known to those of ordinary skill in the art may be employed in the pharmaceutical compositions of this invention, the type of carrier can vary depending on the mode of administration, which can be as described herein for administration of the polypeptide.

DNA or RNA compositions can be administered once or multiple times as needed to induce the desired response. Multiple administrations can be administered, for example, bi-weekly, weekly, bi-monthly, monthly, or more or less often, as needed, for a time period sufficient to achieve the desired response.

Nucleic acids can be administered in solution (e.g., a phosphate-buffered saline solution) by injection, usually by an intravenous, subcutaneous or intramuscular route. Dosages depend on the route of administration and can be readily determined by one of skill in the art.

In some embodiments, cells of an individual are genetically modified using modification techniques, such as CRISPR/CAS to cause expression of HHIP soluble polypeptides. For example, in some embodiments, cells (e.g., lung cells) from an individual are genetically modified using a CRISPR-Cas9 to express an HHIP soluble.

The HHIP soluble polypeptides, or polynucleotides encoding the polypeptides, described herein can be used to treat, ameliorate, or prevent inflammation, for example in lungs. Exemplary inflammatory disorders that can be ameliorated by the HHIP soluble polypeptides include, but are not limited to, chronic obstructive pulmonary disease (COPD), asthma, fibrotic disorders, such as airway fibrosis, idiopathic pulmonary fibrosis, non-specific interstitial pneumonia, post-infectious lung fibrosis, diffuse alveolar damage, collagen-vascular disease associated lung fibrosis, drug-induced lung fibrosis, silicosis, asbestos-related lung fibrosis, respiratory bronchiolitis, follicular bronchiolitis, respiratory bronchiolitis, interstitial lung disease, desquamative interstitial fibrosis, cryptogenic organizing pneumonia, and chronic hypersensitivity pneumonia and graft-versus host disease of a lung transplant. Accordingly, the polypeptides and pharmaceutical compositions described herein can be administered to a human having or suspected of having one of the above-listed diseases in an appropriate dosage to ameliorate or treat one of the disease or at least one symptom thereof.

Without intending to limit the scope of the invention, in some embodiments it is believed that HHIP soluble polypeptides described herein function in part by binding Hedgehog and acting as a “decoy” to prevent Hedgehog from otherwise binding receptors that trigger Hedgehog signaling resulting in inflammation. For example, Hedgehog can induce IL-7 in lung fibroblasts to maintain tissue-resident T cells (TRLs) (e.g., CD45−/CD3+ cells) residence in adventitial regions in proximity to airway epithelia and this mechanism can be inhibited by the presence of soluble HHIP.

The following examples are offered to illustrate, but not to limit the claimed invention.

### Example

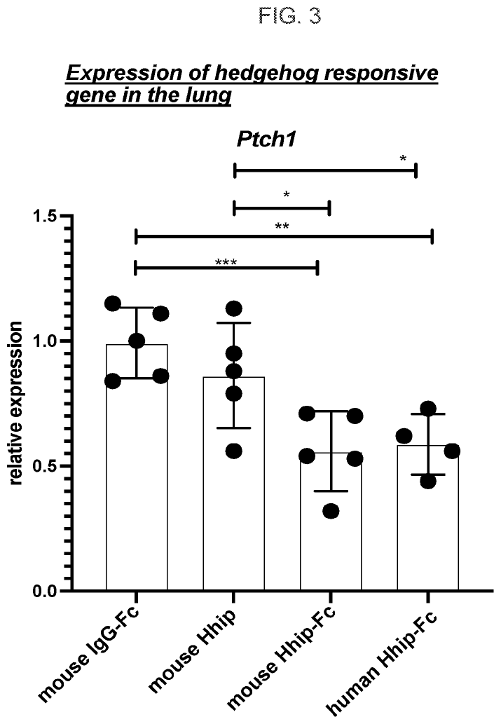

To evaluate the efficacy of the full length HHIP-Fc (Seq ID NO:5, Version 1) in blocking hedgehog activation in the lung, we administered 20 μg of HHIP-Fc intranasally in mice and assayed for the expression of hedgehog-activated gene, Ptch1. RNA transcript analysis showed that HHIP-Fc (box bar in FIG. 3) was able to significantly reduce the expression of Ptch1 in the lung.

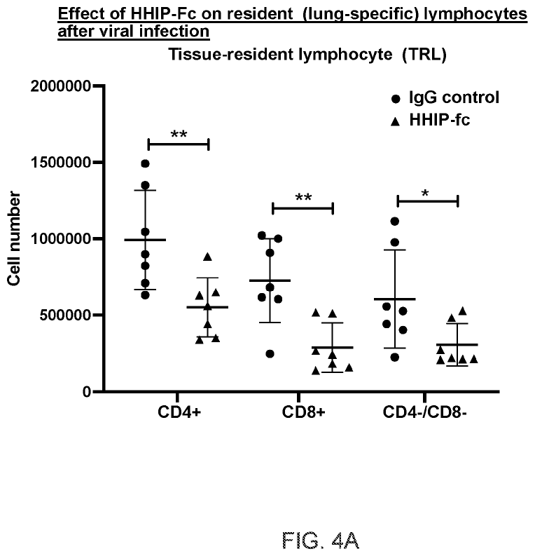

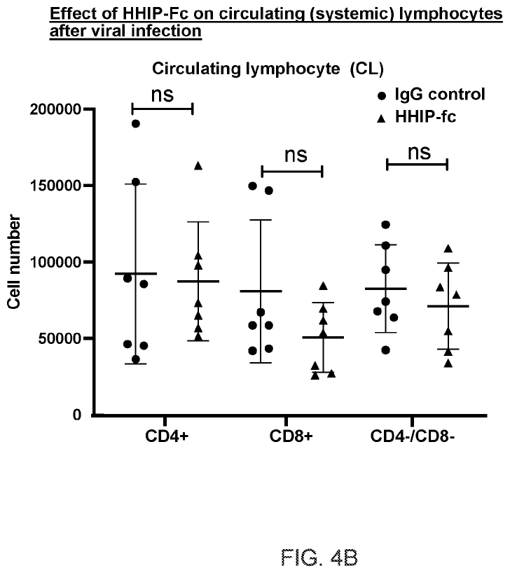

To evaluate the effect of HHIP-Fc on inflammation in the lung, we administered 20 μg of HHIP-Fc intranasally followed by a mild respiratory infection with adenovirus to induce lung inflammation. Flow cytometry analysis 7 days after viral induction (FIG. 4) demonstrated that the administration of HHIP-Fc reduced the accumulation of all the subsets (CD4+, CD8+, CD4−/CD8−) of tissue resident lymphocytes (TRLs), including TRLs that express the cytotoxic cytokine, IFNg.

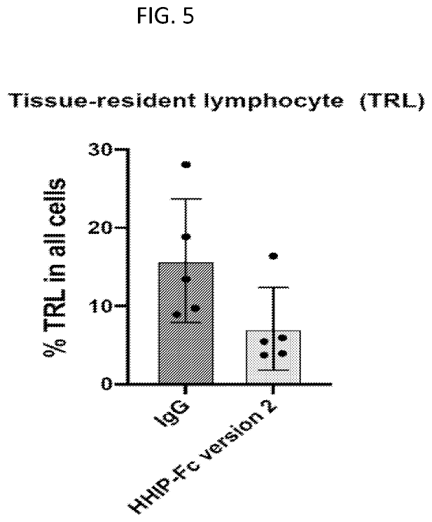

To evaluate the effect of a truncated version of HHIP-Fc (Seq ID NO:6, Version 2), we administered 20 μg of HHIP-Fc version 2 intranasally followed by a mild respiratory infection with adenovirus to induce lung inflammation. Flow cytometry analysis 7 days after viral induction (FIG. 5) demonstrated that the administration of the truncated HHIP-Fc reduced the accumulation of TRLs in the lung.

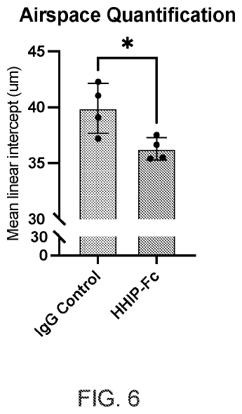

To evaluate the effect of HHIP-Fc administration in an animal model of COPD/emphysema viral exacerbation, we administered 20 μg of HHIP-Fc intranasally on a weekly basis over 6 weeks in mice missing one copy of the Hhip allele (Hhip haploinsufficiency, or Hhip^{+/-}). Hhip^{+/-} animals infected with adenovirus develops airspace enlargement (as measured by mean linear intercept, or MLI) seen in emphysema patients on histologic analysis, which is rescued with the administration of HHIP-Fc (FIG. 6).

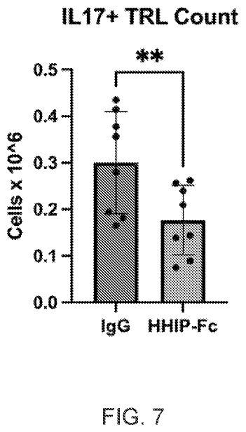

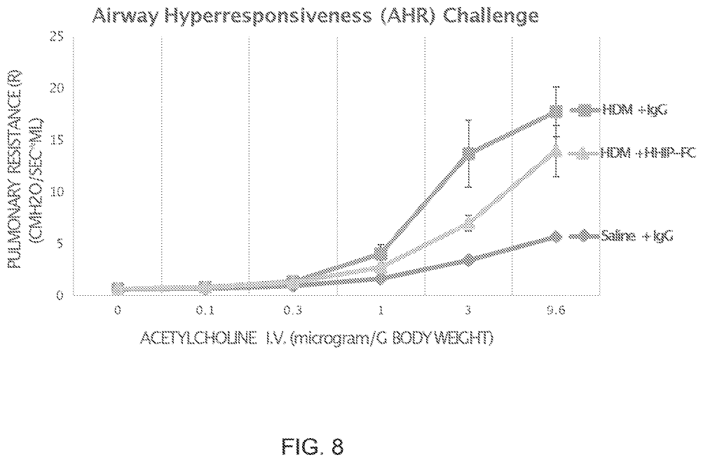

To evaluate the effect of HHIP-Fc administration in an animal model of allergic inflammation relevant to asthma, we administered 20 μg of HHIP-Fc intranasally prior to challenge with house dust mite (HDM) to induce allergic inflammation seen in asthmatic patients. Flow cytometry analysis 3 days after HDM challenge demonstrated reduction of IL17+ TRLs previously implicated in asthma (FIG. 7). Furthermore, measurement of airway resistance (R) in mice demonstrated that HHIP-Fc significantly reduced airway hyperresponsiveness in response to acetylcholine challenge (FIG. 8), which is a pathognomonic feature of asthma.

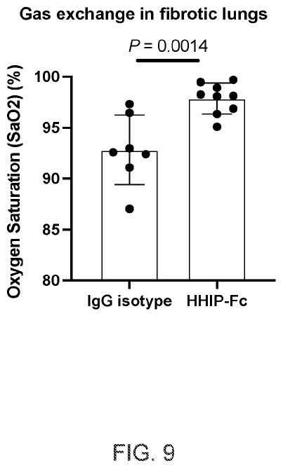

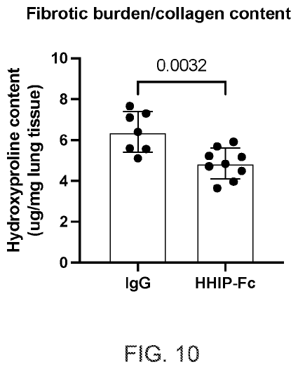

To evaluate the effect of HHIP-Fc administration in an animal model of lung fibrosis, we administered 20 μg of HHIP-Fc intranasally on a weekly basis over 4 weeks in bleomycin-injured mice with established lung fibrosis. Animals treated with HHIP-Fc demonstrated significantly improved gas exchange as measured by oxygen saturation (SaO2) (FIG. 9) as well as fibrotic burden as measured by hydroxyproline content (FIG. 10).

Many modifications and variations of this invention can be made without departing from its spirit and scope, as will be apparent to those skilled in the art. The specific embodiments described herein are offered by way of example only and are not meant to be limiting in any way.

All documents (for example, patents, patent applications, books, journal articles, or other publications) cited herein are incorporated by reference in their entirety and for all purposes, to the same extent as if each individual document was specifically and individually indicated to be incorporated by reference in its entirety for all purposes. To the extent such documents incorporated by reference contradict the disclosure contained in the specification, the specification is intended to supersede and/or take precedence over any contradictory material.

SEQUENCES

SEQ ID NO: 1 Human HHIP (Gly190-Arg610):
GPASNYLDQMEEYDK VEEISRKHKHNCFCIQEVVSGLRQPVGALHSGDGSQRLFILEKE
GYVKILTPEGEIFKEPYLDIHKLVQSGIKGGDERGLLSLAFHPNYKKNGKLYVSYTTNQE
RWAIGPHDHILRVVEYTVSRKNPHQVDLRTARVFLEVAELHRKHLGGQLLFGPDGFLYI
ILGDGMITLDDMEEMDGLSDFTGSVLRLDVDTDMCNVPYSIPRSNPHENSTNQPPEVFA
HGLHDPGRCAVDRHPTDININLTILCSDSNGKNRSSARILQIIKGKDYESEPSLLEFKPFSN
GPLVGGFVYRGCQSERLYGSYVFGDRNGNFLTLQQSPVTKQWQEKPLCLGTSGSCRGY
FSGHILGFGEDELGEVYILSSSKSMTQTHNGKLYKIVDPKRPLMPEECRATVQPAQTLTS
ECSR
SEQ ID NO: 2 Human HHIP (Phe18-Asp670):
FFEGDAKFGERNEGSGARRRRCLNGNPPKRLKRRDRRMMSQLELLSGGEMLCGGFYPR
LSCCLRSDSPGLGRLENKIFSVTNNTECGKLLEEIKCALCSPHSQSLFHSPEREVLERDLV
LPLLCKDYCKEFFYTCRGHIPGFLOTTADEFCFYYARKDGGLCFPDFPRKQVRGPASNY
LDQMEEYDKVEEISRKHKHNCFCIQEVVSGLRQPVGALHSGDGSQRLFILEKEGYVKILT
PEGEIFKEPYLDIHKLVQSGIKGGDERGLLSLAFHPNYKKNGKLYVSYTTNQERWAIGPH
DHILRVVEYTVSRKNPHQVDLRTARVFLEVAELHRKHLGGQLLFGPDGFLYIILGDGMIT
LDDMEEMDGLSDFTGSVLRLDVDTDMCNVPYSIPRSNPHFNSTNQPPEVFAHGLHDPGR
CAVDRHPTDININLTILCSDSNGKNRSSARILQIIKGKDYESEPSLLEFKPFSNGPLVGGFV
YRGCQSERLYGSYVFGDRNGNFLTLQQSPVTKQWQEKPLCLGTSGSCRGYFSGHILGFG
EDELGEVYILSSSKSMTQTHNGKLYKIVDPKRPLMPEECRATVQPAQTLTSECSRLCRNG
YCTPTGKCCCSPGWEGDFCRTAKCEPACRHGGVCVRPNKCLCKKGYLGPQCEQVD
SEQ ID NO: 3: GSGGGGSGGGG
SEQ ID NO: 4: Fc sequence:
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGK
SEQ ID NO: 5 Version 1 Human HHIP (Phe18-Asp670)-GS Linker-hulgG1 Fc:
FFEGDAKFGERNEGSGARRRRCLNGNPPKRLKRRDRRMMSQLELLSGGEMLCGGFYPR
LSCCLRSDSPGLGRLENKIFSVTNNTECGKLLEEIKCALCSPHSQSLFHSPEREVLERDLV
LPLLCKDYCKEFFYTCRGHIPGFLQTTADEFCFYYARKDGGLCFPDFPRKQVRGPASNY
LDQMEEYDKVEEISRKHKHNCFCIQEVVSGLRQPVGALHSGDGSQRLFILEKEGYVKILT
PEGEIFKEPYLDIHKLVQSGIKGGDERGLLSLAFHPNYKKNGKLYVSYTTNQERWAIGPH
DHILRVVEYTVSRKNPHQVDLRTARVFLEVAELHRKHLGGQLLFGPDGFLYIILGDGMIT
LDDMEEMDGLSDFTGSVLRLDVDTDMCNVPYSIPRSNPHFNSTNQPPEVFAHGLHDPGR
CAVDRHPTDININLTILCSDSNGKNRSSARILQIIKGKDYESEPSLLEFKPFSNGPLVGGFV
YRGCQSERLYGSYVFGDRNGNFLTLQQSPVTKQWQEKPLCLGTSGSCRGYFSGHILGFG
EDELGEVYILSSSKSMTQTHNGKLYKIVDPKRPLMPEECRATVQPAQTLTSECSRLCRNG
YCTPTGKCCCSPGWEGDFCRTAKCEPACRHGGVCVRPNKCLCKKGYLGPQCEQVDGS
GGGGSGGGGDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
K
SEQ ID NO: 6 Version 2 Human HHIP (prop)(Gly190-Arg610)-GS
Linker-hulgG1 Fc
GPASNYLDQMEEYDKVEEISRKHKHNCFCIQEVVSGLRQPVGALHSGDGSQRLFILEKE
GYVKILTPEGEIFKEPYLDIHKLVQSGIKGGDERGLLSLAFHPNYKKNGKLYVSYTTNQE
RWAIGPHDHILRVVEYTVSRKNPHQVDLRTARVFLEVAELHRKHLGGQLLFGPDGFLYI
ILGDGMITLDDMEEMDGLSDFTGSVLRLDVDTDMCNVPYSIPRSNPHENSTNQPPEVFA
HGLHDPGRCAVDRHPTDININLTILCSDSNGKNRSSARILQIIKGKDYESEPSLLEFKPFSN
GPLVGGFVYRGCQSERLYGSYVFGDRNGNFLTLQQSPVTKQWQEKPLCLGTSGSCRGY
FSGHILGFGEDELGEVYILSSSKSMTQTHNGKLYKIVDPKRPLMPEECRATVQPAQTLTS
ECSRGGGGSGGGGDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSL
SLSPGK:
SEQ ID NO: 7 Version 3 Human HHIP (prop)(Gly190-Arg610)-GS
Linker-Human HHIP (prop)(Gly 190-Arg610)-hulgG1 Fc:
GPASNYLDQMEEYDK VEEISRKHKHNCFCIQEVVSGLRQPVGALHSGDGSQRLFILEKE
GYVKILTPEGEIFKEPYLDIHKLVQSGIKGGDERGLLSLAFHPNYKKNGKLYVSYTTNQE
RWAIGPHDHILRVVEYTVSRKNPHQVDLRTARVFLEVAELHRKHLGGQLLFGPDGFLYI
ILGDGMITLDDMEEMDGLSDFTGSVLRLDVDTDMCNVPYSIPRSNPHENSTNQPPEVFA
HGLHDPGRCAVDRHPTDININLTILCSDSNGKNRSSARILQIIKGKDYESEPSLLEFKPFSN
GPLVGGFVYRGCQSERLYGSYVFGDRNGNFLTLQQSPVTKQWQEKPLCLGTSGSCRGY
FSGHILGFGEDELGEVYILSSSKSMTQTHNGKLYKIVDPKRPLMPEECRATVQPAQTLTS
ECSRGGGGSGGGGSGGGGSASGPASNYLDQMEEYDKVEEISRKHKHNCFCIQEVVSGL
RQPVGALHSGDGSQRLFILEKEGYVKILTPEGEIFKEPYLDIHKLVQSGIKGGDERGLLSL
AFHPNYKKNGKLYVSYTTNQERWAIGPHDHILRVVEYTVSRKNPHQVDLRTARVFLEV
AELHRKHLGGQLLFGPDGFLYIILGDGMITLDDMEEMDGLSDFTGSVLRLDVDTDMCN
VPYSIPRSNPHFNSTNQPPEVFAHGLHDPGRCAVDRHPTDININLTILCSDSNGKNRSSAR
ILQIIKGKDYESEPSLLEFKPFSNGPLVGGFVYRGCQSERLYGSYVFGDRNGNFLTLQQSP
VTKQWQEKPLCLGTSGSCRGYFSGHILGFGEDELGEVYILSSSKSMTQTHNGKLYKIVD
PKRPLMPEECRATVQPAQTLTSECSRGGGGSGGGGDKTHTCPPCPAPELLGGPSVFLFPP
KPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKN
QVSLTCLVKGFYPSDIA VEWESNGQPENNYKTTPPVLDSDGSFFLYSKLTVDKSRWQQG
NVFSCSVMHEALHNHYTQKSLSLSPGK
SEQ ID NO: 8 (linker between two soluble portions): GGGGSGGGGSGGGGSAS

What is claimed is:

1. A polypeptide comprising at least one soluble Hedgehog-binding portion of a human Hedgehog Interacting Protein (HHIP) linked to a molecule that enhances blood half-life of the polypeptide.

2. The polypeptide of claim 1, wherein the portion comprises SEQ ID NO:1 or an amino acid sequence at least 90 or 95 or 99% identical thereto.

3. The polypeptide of claim 1, wherein the portion comprises SEQ ID NO:2 or an amino acid sequence at least 90 or 95 or 99% identical thereto.

4. The polypeptide of any one of claims 1-3, wherein the polypeptide comprises two soluble Hedgehog-binding portions of a HHIP.

5. The polypeptide of claim 4, wherein each of the two soluble Hedgehog-binding portions comprise SEQ ID NO:1 or an amino acid sequence at least 90 or 95 or 99% identical thereto.

6. The polypeptide of claim 4, wherein the two soluble Hedgehog-binding portions are linked to each other via a linker sequence.

7. The polypeptide of claim 6, wherein the linker sequence comprises SEQ ID NO:8.

8. The polypeptide of any one of claims 1-7, wherein the at least one soluble Hedgehog-binding portion is linked via an amino acid linker to the molecule.

9. The polypeptide of claim 8, wherein the amino acid linker comprises glycine and serine.

10. The polypeptide of claim 9, wherein the amino acid linker comprises SEQ ID NO:3.

11. The polypeptide of claim 10, wherein the polypeptide comprises SEQ ID NO:7 or an amino acid sequence at least 90 or 95 or 99% identical thereto.

12. The polypeptide of any one of claims 1-9, wherein the molecule is an Fc portion of an antibody.

13. The polypeptide of claim 12, wherein the Fc portion comprises SEQ ID NO:4 or an amino acid sequence at least 90 or 95 or 99% identical thereto.

14. The polypeptide of claim 13, wherein the polypeptide comprises SEQ ID NO: 5 or SEQ ID NO:6 or an amino acid sequence at least 90 or 95 or 99% identical thereto.

15. The polypeptide of any one of claims 1-6, wherein the molecule is polyethylene glycol.

16. A polynucleotide encoding the polypeptide of any one of claims 1-12.

17. An expression cassette or expression vector comprising a promoter operably linked to the polynucleotide of claim 16.

18. A dimer comprising two polypeptides, each polypeptide comprising at least one soluble Hedgehog-binding portion of a human Hedgehog Interacting Protein (HHIP) linked to an Fc portion of an antibody, wherein the Fc portions of the two polypeptides are linked by one or more disulfide bond.

19. A host cell comprising the expression cassette or expression vector of claim 17.

20. A method of inhibiting Hedgehog signaling in a human in need thereof, the method comprising administering the polypeptide of any one of claims 1-15, or the dimer of claim 18, or a nucleic acid comprising the expression cassette of claim 17 to the human in an amount sufficient to inhibit Hedgehog signaling.

21. The method of claim 20, wherein the administering comprises administering the polypeptide or nucleic acid orally, intravenously, or via inhalation nasally or orally.

22. The method of claim 20 or 21, wherein the human has inflammation in the lungs and administration of the polypeptide or nucleic acid reduces the inflammation.

23. The method of claim 22, wherein the human has chronic obstructive pulmonary disease (COPD), pulmonary fibrosis, asthma, lung cancer, graft-versus host disease of a lung transplant, follicular bronchiolitis or interstitial lung disease.

24. An isolated polypeptide comprising an amino acid sequence having at least 90% amino acid sequence identity to the amino acid sequence of SEQ ID NO: 2 linked to a molecule that enhances blood half-life of the polypeptide.

25. An isolated nucleic acid encoding the isolated polypeptide of claim 24.

26. An expression cassette or vector comprising the isolated nucleic acid of claim 25 operably linked to a promoter.

27. A host cell comprising the expression vector of claim 26.

28. The isolated polypeptide of claim 24, wherein the polypeptide comprises an amino acid sequence having at least 90, 95 or 99% amino acid sequence identity to the amino acid sequence of SEQ ID NO: 5.

29. The isolated polypeptide of claim 24, wherein the polypeptide binds to human Hedgehog protein.

30. The isolated polypeptide of claim 24, wherein the molecule comprises at least one of an Fc portion of an antibody and polyethylene glycol.

31. The isolated polypeptide of claim 30, wherein the Fc portion is an IgG Fc portion.

32. A method of treating a lung disease in a subject, the method comprising administering an isolated polypeptide comprising at least one soluble Hedgehog-binding portion of a human Hedgehog Interacting Protein (HHIP) linked to a molecule that enhances blood half-life of the polypeptide.

33. The method of claim 32, wherein the lung disease is one or more of chronic lung disease, chronic obstructive pulmonary disease (COPD), pulmonary fibrosis, asthma, lung cancer, graft-versus host disease of a lung transplant, follicular bronchiolitis or interstitial lung disease.

34. The method of claim 33, wherein (1) the soluble Hedgehog-binding portion comprises the sequence of SEQ ID NO: 1 or an amino acid sequence having at least 90% identity to the sequence of SEQ ID NO: 1; or (2) the soluble Hedgehog-binding portion comprises the sequence of SEQ ID NO:2 or an amino acid sequence having at least 90% identity to the sequence of SEQ ID NO: 2.

35. The method of claim 32, wherein the isolated polypeptide comprises two soluble Hedgehog-binding portions of a HHIP, optionally wherein each of the two soluble Hedgehog-binding portions comprise the sequence of SEQ ID NO: 1 or an amino acid sequence having at least 90% identity to the sequence of SEQ ID NO: 1, optionally wherein the two soluble Hedgehog-binding portions are linked to each other via a linker sequence, and optionally wherein the linker sequence comprises the sequence of SEQ ID NO: 8.

36. The method of claim 32, wherein the at least one soluble Hedgehog-binding portion is linked to the molecule via an amino acid linker, optionally wherein the amino acid linker comprises glycine and serine, or the sequence of SEQ ID NO: 3.

37. The method of claim 32, wherein the molecule comprises an Fc portion of an antibody, optionally wherein the Fc portion comprises the sequence of SEQ ID NO:4 or an amino acid sequence having at least 90% identity to the sequence of SEQ ID NO: 4.

38. The method of claim 37, wherein the antibody is a dimer and the Fc portion of each antibody is linked by one or more disulfide bonds.

39. The method of claim 32, wherein the molecule comprises polyethylene glycol.

40. The method of claim 32, wherein the isolated polypeptide comprises an amino acid sequence having at least 90, 95, or 99% amino acid sequence identity to the amino acid sequence of SEQ ID NO: 5, 6 or 7.

41. A method of reducing tissue-resident lymphocytes (TRLs) in a subject with inflammation, comprising administering an isolated polypeptide comprising at least one soluble Hedgehog-binding portion of a human Hedgehog Interacting Protein (HHIP) linked to a molecule that enhances blood half-life of the polypeptide.

42. A method of manufacturing an isolated polypeptide, comprising obtaining the host cell of claim 27, and manufacturing the isolated polypeptide.

43. A pharmaceutical composition comprising the isolated polypeptide of claim 24 and a pharmaceutically acceptable carrier.


SEQUENCE LISTING (8 sequences)

SEQ ID NO: 1 - protein, 421 aa, synthetic construct
  REGION 1..421: note = Description of sequence: Human HHIP (Gly190-Arg610)
GPASNYLDQMEEYDKVEEISRKHKHNCFCIQEVVSGLRQPVGALHSGDGSQRLFILEKEGYVKILTPEGEIFKEPYLDIHKLVQSGIKGGDERGLLSLAFHPNYKKNGKLYVSYTTNQERWAIGPHDHILRVVEYTVSRKNPHQVDLRTARVFLEVAELHRKHLGGQLLFGPDGFLYIILGDGMITLDDMEEMDGLSDFTGSVLRLDVDTDMCNVPYSIPRSNPHFNSTNQPPEVFAHGLHDPGRCAVDRHPTDININLTILCSDSNGKNRSSARILQIIKGKDYESEPSLLEFKPFSNGPLVGGFVYRGCQSERLYGSYVFGDRNGNFLTLQQSPVTKQWQEKPLCLGTSGSCRGYFSGHILGFGEDELGEVYILSSSKSMTQTHNGKLYKIVDPKRPLMPEECRATVQPAQTLTSECSR

SEQ ID NO: 2 - protein, 653 aa, synthetic construct
  REGION 1..653: note = Description of sequence: Human HHIP (Phe18-Asp670)
FFEGDAKFGERNEGSGARRRRCLNGNPPKRLKRRDRRMMSQLELLSGGEMLCGGFYPRLSCCLRSDSPGLGRLENKIFSVTNNTECGKLLEEIKCALCSPHSQSLFHSPEREVLERDLVLPLLCKDYCKEFFYTCRGHIPGFLQTTADEFCFYYARKDGGLCFPDFPRKQVRGPASNYLDQMEEYDKVEEISRKHKHNCFCIQEVVSGLRQPVGALHSGDGSQRLFILEKEGYVKILTPEGEIFKEPYLDIHKLVQSGIKGGDERGLLSLAFHPNYKKNGKLYVSYTTNQERWAIGPHDHILRVVEYTVSRKNPHQVDLRTARVFLEVAELHRKHLGGQLLFGPDGFLYIILGDGMITLDDMEEMDGLSDFTGSVLRLDVDTDMCNVPYSIPRSNPHFNSTNQPPEVFAHGLHDPGRCAVDRHPTDININLTILCSDSNGKNRSSARILQIIKGKDYESEPSLLEFKPFSNGPLVGGFVYRGCQSERLYGSYVFGDRNGNFLTLQQSPVTKQWQEKPLCLGTSGSCRGYFSGHILGFGEDELGEVYILSSSKSMTQTHNGKLYKIVDPKRPLMPEECRATVQPAQTLTSECSRLCRNGYCTPTGKCCCSPGWEGDFCRTAKCEPACRHGGVCVRPNKCLCKKGYLGPQCEQVD

SEQ ID NO: 3 - protein, 11 aa, synthetic construct
  REGION 1..11: note = Description of sequence: Linker
GSGGGGSGGGG

SEQ ID NO: 4 - protein, 227 aa, synthetic construct
  REGION 1..227: note = Description of sequence: Fc sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 891 aa, synthetic construct
  REGION 1..891: note = Description of sequence: Version 1 Human HHIP (Phe18-Asp670)-GS Linker-huIgG1 Fc
FFEGDAKFGERNEGSGARRRRCLNGNPPKRLKRRDRRMMSQLELLSGGEMLCGGFYPRLSCCLRSDSPGLGRLENKIFSVTNNTECGKLLEEIKCALCSPHSQSLFHSPEREVLERDLVLPLLCKDYCKEFFYTCRGHIPGFLQTTADEFCFYYARKDGGLCFPDFPRKQVRGPASNYLDQMEEYDKVEEISRKHKHNCFCIQEVVSGLRQPVGALHSGDGSQRLFILEKEGYVKILTPEGEIFKEPYLDIHKLVQSGIKGGDERGLLSLAFHPNYKKNGKLYVSYTTNQERWAIGPHDHILRVVEYTVSRKNPHQVDLRTARVFLEVAELHRKHLGGQLLFGPDGFLYIILGDGMITLDDMEEMDGLSDFTGSVLRLDVDTDMCNVPYSIPRSNPHFNSTNQPPEVFAHGLHDPGRCAVDRHPTDININLTILCSDSNGKNRSSARILQIIKGKDYESEPSLLEFKPFSNGPLVGGFVYRGCQSERLYGSYVFGDRNGNFLTLQQSPVTKQWQEKPLCLGTSGSCRGYFSGHILGFGEDELGEVYILSSSKSMTQTHNGKLYKIVDPKRPLMPEECRATVQPAQTLTSECSRLCRNGYCTPTGKCCCSPGWEGDFCRTAKCEPACRHGGVCVRPNKCLCKKGYLGPQCEQVDGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 657 aa, synthetic construct
  REGION 1..657: note = Description of sequence: Version 2 Human HHIP (prop)(Gly190-Arg610)-GS Linker-huIgG1 Fc
GPASNYLDQMEEYDKVEEISRKHKHNCFCIQEVVSGLRQPVGALHSGDGSQRLFILEKEGYVKILTPEGEIFKEPYLDIHKLVQSGIKGGDERGLLSLAFHPNYKKNGKLYVSYTTNQERWAIGPHDHILRVVEYTVSRKNPHQVDLRTARVFLEVAELHRKHLGGQLLFGPDGFLYIILGDGMITLDDMEEMDGLSDFTGSVLRLDVDTDMCNVPYSIPRSNPHFNSTNQPPEVFAHGLHDPGRCAVDRHPTDININLTILCSDSNGKNRSSARILQIIKGKDYESEPSLLEFKPFSNGPLVGGFVYRGCQSERLYGSYVFGDRNGNFLTLQQSPVTKQWQEKPLCLGTSGSCRGYFSGHILGFGEDELGEVYILSSSKSMTQTHNGKLYKIVDPKRPLMPEECRATVQPAQTLTSECSRGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 1095 aa, synthetic construct
  REGION 1..1095: note = Description of sequence: Version 3 Human HHIP (prop)(Gly190-Arg610)-GS Linker-Human HHIP (prop)(Gly190-Arg610)-huIgG1 Fc
GPASNYLDQMEEYDKVEEISRKHKHNCFCIQEVVSGLRQPVGALHSGDGSQRLFILEKEGYVKILTPEGEIFKEPYLDIHKLVQSGIKGGDERGLLSLAFHPNYKKNGKLYVSYTTNQERWAIGPHDHILRVVEYTVSRKNPHQVDLRTARVFLEVAELHRKHLGGQLLFGPDGFLYIILGDGMITLDDMEEMDGLSDFTGSVLRLDVDTDMCNVPYSIPRSNPHFNSTNQPPEVFAHGLHDPGRCAVDRHPTDININLTILCSDSNGKNRSSARILQIIKGKDYESEPSLLEFKPFSNGPLVGGFVYRGCQSERLYGSYVFGDRNGNFLTLQQSPVTKQWQEKPLCLGTSGSCRGYFSGHILGFGEDELGEVYILSSSKSMTQTHNGKLYKIVDPKRPLMPEECRATVQPAQTLTSECSRGGGGSGGGGSGGGGSASGPASNYLDQMEEYDKVEEISRKHKHNCFCIQEVVSGLRQPVGALHSGDGSQRLFILEKEGYVKILTPEGEIFKEPYLDIHKLVQSGIKGGDERGLLSLAFHPNYKKNGKLYVSYTTNQERWAIGPHDHILRVVEYTVSRKNPHQVDLRTARVFLEVAELHRKHLGGQLLFGPDGFLYIILGDGMITLDDMEEMDGLSDFTGSVLRLDVDTDMCNVPYSIPRSNPHFNSTNQPPEVFAHGLHDPGRCAVDRHPTDININLTILCSDSNGKNRSSARILQIIKGKDYESEPSLLEFKPFSNGPLVGGFVYRGCQSERLYGSYVFGDRNGNFLTLQQSPVTKQWQEKPLCLGTSGSCRGYFSGHILGFGEDELGEVYILSSSKSMTQTHNGKLYKIVDPKRPLMPEECRATVQPAQTLTSECSRGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 17 aa, synthetic construct
  REGION 1..17: note = Description of sequence: linker between two soluble portions
GGGGSGGGGSGGGGSAS